(19) United States
(12) Patent Application Publication
(10) Pub. No.: US 2003/0133938 A1
(43) Pub. Date: Jul. 17, 2003

(54) Antibody-avidin fusion proteins as cytotoxic drugs

(76) Inventors: Manuel L. Penichet, Los Angeles, CA (US); Sherie L. Morrison, Los Angeles, CA (US); Seung-Uon Shin, Los Angeles, CA (US); Patrick P. Ng, Los Angeles, CA (US)

Correspondence Address: David J. Oldenkamp, Esq., Shapiro, Borenstein & Dupont LLP, Suite 700, 233 Wilshire Boulevad, Santa Monica, CA, 90401, US

(21) Appl. No.: 10/051,652

(22) Filed: Jan. 15, 2002

(51) Int. Cl. A61K 39/395

(52) U.S. Cl. 424/178.1

(57) ABSTRACT

Methods and compositions for inducing apoptosis and/or inhibiting proliferation of cells. The method includes exposing the cells to a cytotoxic agent which is made up of a targeting moiety and an avidin moiety wherein the targeting moiety is capable of binding to one or more receptors located on the cells. The invention is based on the discovery that attaching an avidin moiety to non-toxic targeting moieties produces a cytotoxic agent which can be used to treat tumor cells both in vivo and in vitro. The present cytotoxic agent eliminates the use of biotinylated toxic drugs which previously have been conjugated to antibody-avidin targeting vehicles.

This invention was made with Government support under Grant No. CA86915, awarded by the National Institutes of Health. The Government has certain rights in this invention.

### BACKGROUND OF THE INVENTION

1. Field of the Invention

The present invention relates generally to compositions and methods for treating cells to cause apoptosis and/or inhibit proliferation. More particularly, the present invention involves the discovery that non-toxic targeting moieties can be converted into cytotoxic agents that cause apoptosis and/or inhibit proliferation in a wide variety of cell populations.

2. Description of Related Art

The publications and other reference materials referred to herein to describe the background of the invention and to provide additional detail regarding its practice are hereby incorporated by reference. For convenience, the reference materials are numerically referenced and grouped in the appended bibliography.

There has been intense interest in the medical community to develop pharmaceutical compositions that are able to deliver drugs to specifically targeted cells. Such compositions have typically included a targeting or transport moiety that is conjugated to the drug or diagnostic agent of interest. Antibodies that target antigenic receptors located on cell surfaces have been particularly popular. These antibodies are capable of transporting a wide variety of drugs and diagnostic agents to the cell surface. In many cases, the entire antibody-drug conjugate undergoes receptor-mediated endocytosis into the cell.

The bond between avidin and biotin is one of the highest affinity binding reactions found in nature with a molar dissociation constant of 10⁻¹⁵ M and a t_{1/2} of ligand dissociation of 89 days (10). Avidin is a 64,000 dalton homotetramer glycoprotein that has been administered to humans in large concentrations without untoward effects (11). Each 16,000 dalton monomer of avidin contains a high-affinity binding site for biotin which is a water soluble vitamin. The avidin cDNA gene was cloned in 1987 so that avidin has been produced routinely using recombinant DNA technology (12).

The avidin-biotin linkage has been a natural choice for use in connecting targeting antibodies to a wide variety of drugs and diagnostic agents. Typically, avidin is first attached to the antibody to form an antibody-avidin targeting vehicle. This targeting vehicle is then reacted with a drug or diagnostic agent that has been previously biotinylated. Although the avidin may be chemically conjugated with the antibody, the preferred procedure has been to use recombinant DNA technology to genetically engineer a fusion protein that includes both the antibody and avidin (1).

Antibody-avidin fusion proteins have been used to transport a variety of other types of drugs including anti-tumor toxins that are used in cancer treatments (14). For example, biotinylated anti-sense oligonucleotides have been attached to antibody-avidin fusion protein target vehicles to form compositions which are useful in gene therapy (13 and 14). Antibody-avidin fusion proteins have also been used to transport a variety of other types of drugs including anti-tumor toxins that are used in cancer treatments (14).

### SUMMARY OF THE INVENTION

In accordance with the present invention, it was discovered that antibody-avidin proteins are, by themselves, effective cytotoxic agents that cause apoptosis in cells and/or inhibit cell proliferation. We found that the non-toxic anti-receptor antibodies which are used as targeting vehicles can be transformed into cytotoxic agents by fusing the antibodies with avidin. The resulting antibody-avidin complex was found to cause apoptosis and inhibition of cell proliferation in cancer cells. In addition, intrinsic cytotoxic activity of known antibodies, such as Rituxan or Herceptin may be enhanced by fusing them to avidin.

The present invention includes a method for inducing apoptosis in cells. The method involves exposing one or more cells to a cytotoxic agent for a sufficient time and at a sufficient temperature to induce apoptosis. The cytotoxic agent, in accordance with the discoveries of the present invention, includes a targeting moiety and an avidin moiety wherein the targeting moiety is capable of binding to one or more receptors located on the cells. The present invention specifically requires that a biotinylated drug not be included as part of the cytotoxic agent. The method of the present invention is particularly well suited for treating both liquid and solid tumor cells and especially those which are cancerous. The method may be used to treat cell populations located both in vivo and in vitro.

The present invention also includes methods for inhibiting the proliferation of a proliferating cell population such as a liquid or solid tumor. It was discovered that cytotoxic agents in accordance with the present invention were effective not only in inducing apoptosis, but also effective in inhibiting proliferation of cancerous cell populations. The method for inhibiting proliferation of tumor cells may also be used both in vivo and in vitro.

The present invention also covers compositions for use in treating cells to induce apoptosis and/or inhibit cell proliferation. The composition includes a cytotoxic agent having a targeting moiety and an avidin moiety wherein the targeting moiety is capable of binding to one or more receptors located on the cell surface. The composition further includes a pharmaceutically acceptable carrier. It should be noted that the cytotoxic agent specifically does not include a biotinylated drug attached to the avidin moiety. The compositions of the present invention are intended for use in the above-described methods for inducing apoptosis and inhibiting proliferation in specific cell populations.

The methods and compositions of the present invention are well suited for use in treating a wide variety of diseases both in vivo and in vitro wherein apoptosis and/or inhibition of proliferation of a targeted cell population is required. The methods and compositions are particularly well suited for treating cancerous cells which over express growth factor receptors.

The above described and many other features and attendant advantages of the present invention will become better understood by reference to the following detailed description when taken in conjunction with the accompanying drawings.

### BRIEF DESCRIPTION OF THE DRAWINGS

FIG. 1A is a diagrammatic representation of an exemplary cytotoxic agent in accordance with the present invention. The cytotoxic agent includes an antibody targeting moiety fused to an avidin moiety which is made up of two avidin molecules.

FIG. 1B is a diagrammatic representation of the dimeric form of the cytotoxic agent shown in FIG. 1A. The cytotoxic agent is believed to form into a dimer in solution.

FIG. 2A shows the results of tests where a rat myeloma cell, Y3-Ag 1.2.3, was treated with anti-TfR IgG3-C_{H}3-Av (▪), anti-dansyl IgG3-C_{H}3-Av (□), anti-TfR IgG2a (Δ), anti-TfR IgG3(●), or anti-dansyl IgG3(◯) at various concentrations for 24 hours. The cells were then cultured in the presence of [³H]-thymidine for an additional 24 hours, harvested and [³H]-thymidine incorporation read. Each value is the mean of quadruplicate assays expressed as the % control mean (controls are cells treated with buffer alone).

FIG. 2B shows the results of tests conducted on Y3-Ag 1.2.3 cells (▪), rat bladder carcinoma cells, BC47 (●), and rat glioma cells, 9 L (Δ). The cells were treated with various concentrations of anti-TfR IgG3-C_{H}3-Av for 24 hours and processed in the same manner as FIG. 2A.

FIG. 3 shows the results of tests where anti-TfR IgG3 (173 kDa) and anti-TfR IgG3-C_{H}3-Av (200 kDa) for monomer were analyzed by FPLC in 0.5 M NaCl-PBS on two sequential Superose 6columns. The profile of molecular mass standards, dimeric IgA (360 kDa) and monomeric IgG (150 kDa) separated under identical conditions as shown. Fraction size is 1 mL.

FIG. 4 depicts the results of annexin V/propidium iodide staining in flow cytometry that shows anti-rat TfR IgG3-C_{H}3-Av induces apoptosis in rat myeloma cell line Y3-Ag1.2.3. 5×10⁴ Y3Ag1.2.3 cells were incubated with buffer alone (FIG. 4A), or 9 nM of anti-rat TfR IgG3-C_{H}3-Av (FIG. 4B) for 24 hours. The cells were then washed with PBS, stained with Alexa Fluor 488 annexin V and propidium iodide, followed by flow cytometry analysis. The percentage of cells located in each quadrant is shown at the corner.

FIG. 5 depicts the results of DNA fragmentation tests that show anti-rat TfR IgG-C_{H}3-Av induces apoptosis in rat myeloma cell line Y3-Ag1.2.3 detected in flow cytometry. 5×10⁴ Y3Ag1.2.3 cells were incubated with buffer alone (thin line), or 9 nM of anti-rat TfR IgG3-C_{H}3-Av (bold line) for 48 hours. The cells were then fixed and incubated with TdT, BrdUTP and Alexa Fluor 488 dye-labeled anti-BrdU antibody, and analyzed by flow cytometry.

FIG. 6 shows the results of flow cytometry tests that demonstrate the specificity of anti-TfR IgG3-C_{H}3-Av for the TfR expressed on human erythroleukemia cell line K562. 4 μg of anti-dansyl IgG3-C_{H}3-Av (narrow line) or anti-TfR IgG-C_{H}3-Av (bold line) complexed with FITC-biotin were incubated with 10⁶K562 cells for 3 hours on ice. The cells were then washed and incubated for an additional 1 hour on ice, followed by flow cytometry analysis. The level of binding by anti-dansyl IgG3-C_{H}3-Av-b-FITC is similar to that of b-FITC or cells treated with buffer alone (data not shown).

FIG. 7 shows the results of tests that demonstrate the antiproliferative effect of anti-human TfR-avidin fusion protein on human erythroleukemia cell line. K562 cells were treated with buffer (A), 104 nM of anti-dansyl IgG3-C_{H}3-Av (B), 104 nM of mouse anti-human TfR IgG1 (C), or 104 nM of anti-human TfR IgG3-C_{H}3-Av (D) for 72 hours. The cells were then cultured in the presence of [³H]-thymidine for another 24 hours before being harvested. The antiproliferative effect of each treatment is calculated by measuring [³H]-thymidine incorporation. Each value is the mean of quadruplicate assays expressed as the % of control mean. The control is cells treated with buffer alone.

FIG. 8 shows the results of tests that demonstrate the dose-dependent antiproliferative effect of anti-human TfR-avidin fusion protein on human erythroleukemia cell line. K562 cells were treated with buffer (A), 25.9 nM (B), 51.9 nM (C), or 104 nM (D) of anti-human TfR IgG3-C_{H}3-Av for 72 hours. The cells were then cultured in the presence of [³H]-thymidine for another 24 hours before harvested. The antiproliferative effect of each treatment is calculated by measuring [³H]thymidine incorporation. Each value is the mean of duplicate assays expressed as the % of control mean. The control is cells treated with buffer alone.

### DETAILED DESCRIPTION OF THE INVENTION

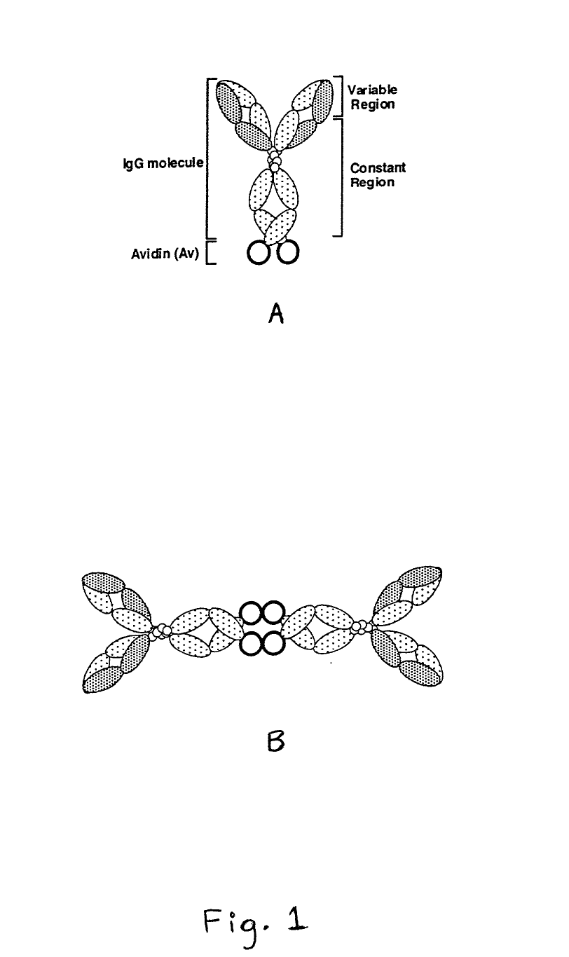

A diagrammatic representation of an exemplary cytotoxic agent in accordance with the present invention is shown in FIG. 1A. The cytotoxic agent includes the variable and constant regions of an IgG antibody and two avidin molecules. Antibody-avidin fusion proteins of the type shown in FIG. 1A have been described previously (1, 3, 13, and 14). Fusion proteins of the type shown in FIG. 1A have previously been used as targeting vehicles which are used to deliver biotinylated drugs to specific cell types. In accordance with the present invention, it was discovered that antibody-avidin fusion proteins of the type shown in FIG. 1A can be used as cytotoxic agents to treat cell populations both in vivo and in vitro to cause apoptosis and/or inhibit cell proliferation.

It is not known why the antibody-avidin fusion proteins of the type shown in FIG. 1A cause apoptosis and antiproliferative activity. Although it is not known why fusing avidin to an antibody causes such a cytotoxic effect, it is believed that the following factors may contribute to the observed apoptosis/antiproliferative activity:

(1) Since avidin is a homotetrameric protein and each antibody-avidin fusion protein (FIG. 1A) contains two molecules of avidin (one genetically fused at the carboxy-terminus of each heavy chain) it is possible that two independent antibody fusion proteins bind to each other through their respective avidins to form a dimeric structure a shown in FIG. 1B. This dimeric structure may contribute to the observed activity. It should be note that monomeric fusion antibodies of the type shown in FIG. 1A are initially produced in accordance with the present invention. It is only after the monomeric fusion antibody is placed in solution that it is possible for the two monomers to join together to form a dimer as shown in FIG. 1B.

(2) The presence of the extended hinge region of the antibody provides spacing and flexibility which may facilitate multiple receptor binding. This may result in stronger receptor binding, signaling modulation, receptor crosslinking, and/or receptor down regulation. These are all mechanisms which may contribute to ligand deprivation resulting in cytostasis and eventually cell death.

(3) It is possible that the presence of avidin in the molecule contributes to confer an optimal antibody confirmation resulting in the observed cytotoxic activity.

(4) It is also possible that after specific binding of the antibody-avidin complex to the cell, the positive charge of avidin may contribute to binding stabilization and the observed antiproliferative activity.

It should be noted that one or more of the above-described points may be causing the observed cytotoxic and antiproliferative activity. Further, the above explanations are only hypotheses which may explain the observed intrinsic cytotoxic/antiproliferative activity of the antibody-avidin fusion proteins in accordance with the present invention.

The fused protein shown in FIG. 1A is exemplary only. For example, any antibody class may be used, including IgG, IgE, IgA, and IgM wherein the antibody has specificity for a cell surface protein or carbohydrate. Exemplary cell surface proteins or carbohydrates include growth factor receptors, transferrin receptors, and insulin receptors. Exemplary growth factor receptors include epidermal growth factor receptor, vascular endothelial growth factor receptor, an insulin-like growth factor receptor, platelet-derived growth factor receptor, transforming growth factor β receptor, fibroblast growth factor receptor, interleukin-2 receptor, interleukin-3 receptor, erythropoietin receptor, nerve growth factor receptor, brain-derived neurotrophic factor receptor, neurotrophinn-3 receptor, and neurotrophin-4 receptor.

In addition to antibodies and antibody fragments, receptor ligands or single chain Fvs (scFv) may be used as the targeting moiety provided that they exhibit specificity for a cell surface protein or carbohydrate. Exemplary non-antibody molecules include receptor ligands such as transferrin, insulin, epidermal growth factors, vascular endothelial growth factor, insulin-like growth factor, platelet-derived growth factor, transforming growth factor β, fibroblast growth factor, interleukin-2, interleukin-3 receptor, erythropoietin, nerve growth factor, brain-derived neurotrophic factor, neurotrophinn-3, and neurotrophin-4, and any scFv molecules specific for cell surface protein and/or growth factor receptors such as transferrin receptors, and insulin receptors. Exemplary growth factor receptors include epidermal growth factor receptors, vascular endothelial growth factor receptor, an insulin-like growth factor receptor, platelet-derived growth factor receptor, transforming growth factor β receptor, fibroblast growth factor receptor, interleukin-2 receptor, interleukin-3 receptor, erythropoietin receptor, nerve growth factor receptor, brain-derived neurotrophic factor receptor, neurotrophinn-3 receptor, and neurotrophin-4 receptor.

As shown in FIG. 1A, avidin molecules are the preferred avidin moiety. However, the avidin moiety may also be made up of avidin analogs such as streptavidin, neutra-avidin, lite-avidin, and neutra-lite avidin. It is preferred, although not necessary that the avidin molecules be fused to the C_{H}3 domain of the constant region. The avidin may be fused to mutated antibodies (mutein) or truncated antibodies wherein the avidin is fused after the hinge or after the C_{H}1 domain.

The targeting moiety (i.e., antibody, receptor ligand or scFB) may be conjugated to the avidin moiety using conventional chemical conjugation techniques. However, it is preferred that the targeting moiety-avidin moiety combination be formed as a fusion protein using recombinant DNA techniques. The methods and procedures for forming antibody-fusion proteins are well known to those skilled in the art. Exemplary procedures for forming antibody-avidin fusion proteins are set forth in references Nos. 1, 14, 15, 16, 17, and 18.

The cytotoxic agents in accordance with the present invention may be used in vivo to treat both liquid and solid tumors. The cytotoxic agent is administered to individuals in the same manner as previously described antibody-avidin fusion proteins which have been conjugated to a biotinylated drug. Pharmaceutically acceptable carriers include any of those commonly used to deliver antibody-avidin-biotinylated drug complexes. Intravenous administration is preferred. Exemplary pharmaceutically acceptable carriers include normal saline by itself or in combination with small amounts of detergent. The appropriate therapeutic dosage will vary widely depending upon the particular tumor or cell population being treated. Typically, therapeutic dosage will range from about 0.001 mg/kg bodyweight to about 1 mg/kg bodyweight.

The cytotoxic agents may also be used to treat cells in vitro. For example, the cytotoxic agents may be used to purge cancer cells during ex vivo expansion of hematopoetic progenitor cells for use as an autograph. When treating cell populations in vitro, it is important that the temperature of the cell population be high enough to allow apoptosis to occur. For example, if the cell population is maintained at a relatively low temperature of around 4° C., most cell populations will not undergo apoptosis. Accordingly, it is important that the incubation temperature during in vitro treatments be sufficiently high to allow apoptosis and/or inhibition of cell proliferation to occur. Preferably, the incubation temperature will be between about 37° C. or close to 37° C.

The cells are exposed to the cytotoxic agents for a sufficient time to cause apoptosis and/or inhibition of proliferation. Exposure times will vary depending upon the concentration of the cytotoxic agent, the particular cell type and whether the exposure is in vivo or in vitro. Exposure times may range from a few hours to a few days or more.

Exemplary cytotoxic agents in accordance with the present invention are as follows:

There are two methods to join avidin to a protein: a chemical conjugation or a genetic fusion (recombinant DNA technology). The following are examples of avidin fusion proteins:

1) An immunoglobulin (Ig) of any class or isotype in which avidin is genetically fused at the end of the C_{H}3 domain (Ig-C_{H}3-Avidin), after the hinge (Ig-H-Avidin), or at the end of the C_{H}1 domain (Ig-C_{H}1-Avidin) of the heavy chain (1,33).

2) An immunoglobulin (Ig) of any class or isotype in which avidin is genetically fused at the beginning (N-terminus) of the heavy chain (34, 35).

3) An immunoglobulin (Ig) of any class or isotype in which avidin is genetically fused at the beginning (N-terminus) or at the end (C-terminus) of the light chain (34, 35).

4) An scFv (developed by phage library technology) specific in which avidin is genetically fused at the beginning (N-terminus) or at the end (C-terminus) of the scFv (25).

5) A ligand such as transferrin in which avidin is genetically fused at the beginning (N-terminus) or at the end (C-terminus) of the ligand.

The following are examples of avidin analogs.

1) Streptavidin (10).

2) Mutated streptavidin with decreased immunogenicity (31).

3) Mutated Avidins: Neutral-avidin, Lite-avidin, Neutra-lite avidin (28).

The following are examples of toxins and chemicals that can be added to the avidin fusion proteins to improve their intrinsic effectiveness (the toxins and chemicals should be previously biotynilated). In the case of toxins an alternative approach is the delivery of the gene encoding for the toxin instead of the toxin itself.

1) Diphtheria toxin (DT) (41).

2) Pseudomonas exotoxin A (PE) (19).

3) The plant toxin ricin (27).

4) The mammalian ribonuclease A (RNase A) (37, 38).

5) The chemicals gemcitabine and arabinoside (29, 30).

6) The chemical adriamycin (42)

The following are examples of cell type/diseases which may be targeted and the specific cell receptor. The targeting agent may be an antibody, an antibody fragment, a scFv, or the ligand fused or chemically conjugated with avidin or an avidin analog.

1) Cancer cells expressing the transferrin receptor (TfR) such as

1.1) Malignant brain tumors (22, 23)

1.2) Colorectal cancer (36, 39)

1.3) Hematopoietic malignancies (20, 21)

2) Cancer cells expressing the CD20 receptor such as B-cell lymphomas (18).

3) Cancer cells expressing one or more members of epidermal growth factor (EGF) receptor family such as HER2/neu.

3.1) Breast cancer (26)

3.2) Ovarian cancer (24)

4) Cancer cells expresing interleukin-2 receptor (IL-2R). Leukemic and lymphomatous cells of T and B cell origin (32, 40).

Examples of practice are as follows:

### EXAMPLE 1

### Inhibition of Cancer Cell Proliferation

Materials and Methods

Antibodies and Antibody Fusion Proteins

Anti-TfR IgG3-C_{H}3-Av fusion proteins in accordance with the present invention were constructed by the substitution of the variable region of anti-dansyl (5-dimethylamino naphthalene 1-sulfonyl chloride) IgG3-C_{H}3-Av fusion heavy chain (1) with the variable region of the heavy chain of anti-rat TfR IgG2a monoclonal antibody OX26 (2). It was expressed with the mouse/human k light chain gene with the variable region of OX26 in the murine myeloma P3X63Ag8.653(3). Recombinant anti-TfR IgG3 containing the variable regions of OX26 and recombinant anti-dansyl IgG3 were used as controls. The antibodies and antibody fusion proteins were purified from culture supernatants using protein G immobilized on Sepharose 4B fast flow (Sigma Chemical Company, St. Louis, Mo.). Purity was assessed by Coomassie blue staining of SDS-PAGE gels. All protein concentrations were determined by the bicinchoninic acid based protein assay (BCA Protein Assay, Pierce Chemical Co., Rockford, Ill.) and ELISA. Purified OX26 was supplied by Dr. William M. Pardridge (UCLA). The murine IgG1 anti-human IgG3 hinge monoclonal antibody HP6050 were obtained from Dr. Robert G. Hamilton (John Hopkins University). Goat anti-human IgG was purchased from ZYMED Laboratories, Inc. (So. San Francisco, Calif.).

Cell Lines

Y3-Ag1.2.3 cells were obtained from Dr. Vernon T. Oi (Stanford University). The cell is a myeloma from the Lou strain of rats that is resistant to azaguanine. The cells synthesizes and secretes a rat k light chain and was originally described in Ref. (4). BC47 is a rat bladder carcinoma provided by Dr. H. Tanaguchi (Keio University, Tokio, Japan). The 9L gliobastoma was provided by Dr. J. Laterra (Johns Hopkins University, Baltimore, Md.). All cells were cultured at 37° C., 5% CO₂ in Dulbecco's Modified Eagle Medium (DMEM) (GIBCO BRL, Grand Island, N.Y.), with 5% calf serum (HyClone, Logan, Utah).

Specific Targeting of Y3-Ag1.2.3 cells

10⁶ Y3-Ag1.2.3 cells were incubated with 5 μg of anti-TfR IgG3-C_{H}3-Av, anti-TfR IgG3, anti-dansyl IgG3 or anti-dansyl IgG3-C_{H}3-Av for 3 hours on ice. Cells were then washed twice and incubated with 20 ml of mouse anti-human kappa light chain-FITC conjugate (BD PharMingen, San Diego, Calif.) for 1 hour. Cells were then washed once, resuspended in 2% paraformaldehyde in PBS, pH 7.4 and analyzed by flow cytometry using a FACScan (Becton-Dickinson, Mountain View, Calif.) equipped with a blue laser excitation of 15 mW at 488 nm.

The ability of anti-TfR IgG3-C_{H}3-Av to specifically bind to the TfR expressed on Y3-Ag1.2.3 cells was examined by flow cytometry. The isotype-matched specificity controls, anti-dansyl IgG3 and anti-dansyl IgG3-C_{H}3-Av, did not bind and showed fluorescence intensity similar to that seen with cells treated with buffer (PBS) alone. In contrast, both anti-TfR IgG3 and anti-TfR IgG3-C_{H}3-Av bound to the cells, with anti-TfR IgG3-C_{H}3-Av treated cells showing stronger fluorescence intensity.

Proliferation Inhibition Assays

Y3-Ag1.2.3 cells (10⁴/well in DMEM 5% CS) were treated with buffer (50 mM Tris base, 150 mM NaCl, pH 7.8) alone, with antibodies, or anti-TfR IgG-C_{H}3-Av in a 96-well plate (Becton Dickinson Labware, Franklin Lakes, N.J.) for 24 or 48 hours at 37° C. In a similar study, BC47 and 9L, which are adherent cell lines, were plated 1 day before treatment at 5×10³ cells/well in DMEM 5% CS. After 24 hours, 4 μCi/mL of [methyl-³H]-thymidine (ICN Biomedicals, Inc., Irvine, Calif.) was added and cells were cultured for an additional 24 hours before being harvested onto glass fiber filters using a 11050 Microo Cell Harvester (Skratron, Norway) and counted in a 1205 Betaplate Liquid Scintillation Counter (WALLAC Inc., Gaithersburg, Md.). The assays mentioned above were conducted in quadruplicate and values expressed as % of the control mean.

Purified antibodies and antibody-avidin fusion proteins were analyzed in 0.5 M NaCl, 20 mM phosphate solution, pH 6.5 using two consecutive analytical Superose® 6 HR 10/30 columns (Amersham Pharmacia Biotech, Piscataway, N.J.) at a flow rate of 0.25 ml/min. The injection volume of 100 ml contained 50 mg of antibody or antibody-avidin fusion proteins. Statistical analysis of the experimental findings was made using a two-tailed Student's t-test. Results were regarded as significant if p values were ≦0.05.

Antiproliferative Effect of Antibody Fusion Proteins on Rat Cancer Cell Lines

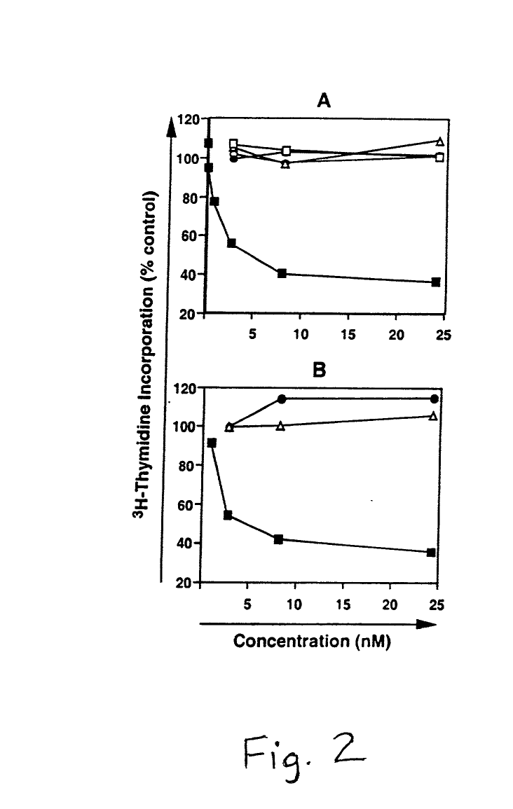

To demonstrate the intrinsic antiproliferative effect of anti-TfR IgG3-C_{H}3-Av on Y3-Ag1.2.3, the cells were incubated with various concentrations of anti-TfR IgG3-C_{H}3-Av or anti-dansyl IgG3-C_{H}3-Av. In addition, recombinant anti-TfR IgG3 and anti-TfR IgG2a (OX26) were included which contain the same variable regions as anti-TfR IgG3-C_{H}3-Av, as well as recombinant anti-dansyl IgG3 (FIG. 2A). The concentration of anti-TfR IgG3-C_{H}3-Av required for 50% inhibition of proliferation (IC50) as measured by thymidine incorporation assay is 4.5 nM. Anti-TfR IgG3, anti-TfR IgG2a, anti-dansyl IgG3 and anti-dansyl IgG3-C_{H}3-Av showed no inhibition of proliferation. Statistical analysis of the highest three concentrations of anti-TfR IgG3-C_{H}3-Av and anti-dansyl IgG3-C_{H}3-Av showed that the anti-TfR IgG3-C_{H}3-Av was a potent inhibitor of proliferation (p≦0.002). Similar results were obtained in two independent studies using the same procedure. This demonstrates that anti-TfR IgG3-C_{H}3-Av exhibits an antiproliferative effect against the rat myeloma that requires both the anti-TfR variable regions and the avidin moiety. Furthermore, this antiproliferative effect was observed only in the rat myeloma cell line, Y3-Ag1.2.3 cells and not in the rat bladder carcinoma, BC47 and rat gliosarcoma, 9L under the conditions tested (FIG. 2B). Anti-dansyl IgG3-C_{H}3-Av, anti-TfR IgG3, anti-dansyl IgG3 and anti-TfR IgG2a did not inhibit the proliferation of BC47 and 9L.

FPLC Analysis of Anti-TfR IgG3-C_{H}3-Av Fusion Protein

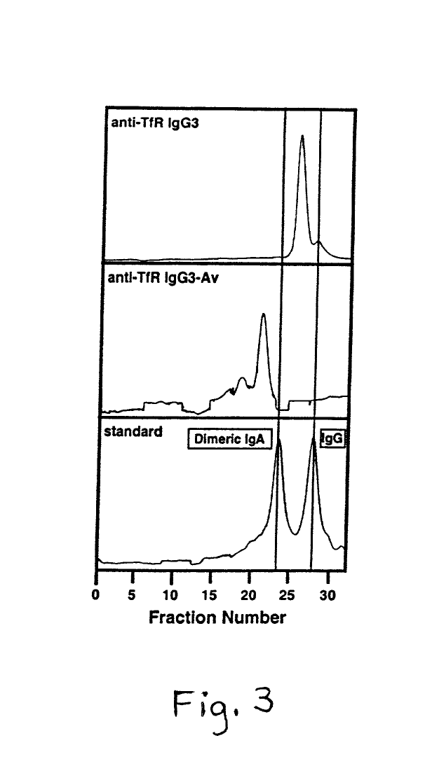

Studies have shown that polymeric TfR specific antibodies have a cytotoxic effect on cancer cells by cross-linking the TfR on cell surface and inhibiting Tf uptake. In a previous study, it was found that anti-dansyl IgG3-C_{H}3-Av exists as dimmers presumably through tetramerization of the avidin moieties (two avidins per fusion protein). Since anti-TfR IgG3-C_{H}3-Av was constructed by changing only the variable regions of anti-dansyl IgG3-C_{H}3-Av, it also is expected to assume a dimeric structure, which may facilitate cross-linking of the TfR on Y3-Ag1.2.3. FPLC analysis (FIG. 3) showed that anti-TfR IgG3 eluted at the position expected given its size (173 kDa). However, anti-TfR IgG3-C_{H}3-Av and anti-dansyl IgG3-C_{H}3-Av (data not shown) appeared to have a molecular mass of approximately 400 kDa, corresponding to a non-covalent dimmer composed of two fusion protein monomers of 200 kDa. This result helps explain why Y3Ag1.2.3 cells treated with anti-TfR IgG3-C_{H}3-Av showed a stronger fluorescent intensity than cells treated with anti-TfR IgG3 in flow cytometry. The fact that anti-TfR IgG3 does not dimerize suggested that it is a non-covalent interaction among the avidin molecules that results in dimerization.

The above examples show that anti-TfR IgG3-C_{H}3-Av has a direct antiproliferationn effect against Y3-Ag1.2.3 cells. Such inhibitory effect can be increased by the addition of deglycosylated Ricin A (b-dgRTA) at an anti-TfR IgG3-C_{H}3-Av concentration of 3 nM. Statistical analysis indicated that there was significant additional inhibition of proliferation by anti-TfR IgG3-C_{H}3-Av plus b-dgRTA compared to anti-TfR IgG3-C_{H}3-Av alone (p=0.0025) when cells were incubated in their presence for 72 hours. Although this difference was significant it was not impressive. The weak, additional cytotoxic effect of b-dgRTA may be attributed to the low concentration of b-dgRTA. This amount may be insufficient to greatly enhance the antiproliferative effect of anti-TfR IgG3-C_{H}3-Av alone. Unfortunately we could not use a higher concentration of b-dgRTA because this commercial product (Sigma Chemical Company, St. Louis, Mo.) is contaminated with some native protein resulting in unspecific cytotoxic effect at higher concentrations. Furthermore, dgRTA lacks the domain on the B chain which facilitates translocation from endocytotic vesicles into the cytosol and, as a result, much of the internalized b-dgRTA may be degraded in the lysosomes. Use of recombinant toxins that lack the ability to enter cells by themselves but contain both cytotoxic as well as the translocation domains may result in more potent antiproliferative agents.

The above examples further demonstrate that anti-TfR IgG3-C_{H}3-Av exists as a noncovalent dimmer. It is believed that the antiproliferative activity of anti-TfR IgG3-C_{H}3-Av may be, at least in part, due to its dimeric structure. For example, it was found that while anti-TfR IgG3 alone did not have any inhibitory activity, anti-TfR IgG3 cross-linked with secondary antibodies exhibited an antiproliferative activity comparable to that of anti-TfR IgG3-C_{H}3-Av.

The examples show a correlation between the valence of anti-TfR antibodies and their growth inhibitory properties. Divalent antibodies such as IgG, increase the rate of TfR internalization and degradation, resulting in decreased TfR receptor expression and cell growth rate in certain cases. However, multivalent antibodies such as IgM (valence=10-12), cause more extensive receptor cross-linking which inhibits internalization, and may even lead to loss of TfR expression in some cells by a mechanism yet to be determined. Cells treated with multivalent antibodies suffer from severe iron deprivation and growth inhibition. Dimeric (tetravalent) anti-TfR IgG3-C_{H}3-Av would be expected to cause a lower level of TfR cross-linking than anti-TfR IgM due to its lower valence and, unlike IgM, anti-TfR IgG3-C_{H}3-Av was able to efficiently deliver biotinylated molecules via receptor mediated endocytosis. The inhibition of growth by anti-TfR IgG3-C_{H}3-Av is likely to reflect a combination of a partial blocking of Tf internalization and receptor down-regulation. This may be aided by the extended hinge region of human IgG3 which provides spacing and flexibility facilitating simultaneous binding to multiple TfRs. In addition, it is possible that the presence of avidin in the molecule may confer an optimal antibody conformation for cytotoxic activity or that the positive charge and glycosylation of avidin may contribute to more stable binding and subsequent internalization.

The examples also show that despite the fact that anti-TfR IgG3-C_{H}3-Av was strongly inhibitory to the growth of Y3-Ag1.2.3 cells, similar treatment did not inhibit the growth of the rat bladder carcinoma cell line (BC47) or the glioblastoma cell line (9L). Low or negative expression of the TfR is unlikely to explain the difference for 9L, which has been used successfully in an anti-TfR immunotoxin study. Instead, these findings are consistent with previous studies showing that hemopoietic cells are generally more sensitive to the antiproliferative effects of anti-TfR monoclonal antibodies than other cell types. These differences may reflect the capacity of individual cell types to respond to iron deprivation. Alternatively, an iron uptake pathway independent of the Tf-TfR system has been demonstrated in a murine cell and it is possible that different cells may vary in their dependence on the Tf-TfR system for iron supply.

A concern is whether there will be non-specific cytotoxicity associated with the in vivo use of anti-TfR IgG3-C_{H}3-Av. However, treatment of mice challenged with SL-2 leukemic cells with 3 mg of anti-mouse TfR IgM, R17 208 twice weekly for up to 4 weeks produced no evidence of gross toxicity or cellular damage. The similar antiproliferative effect seen with R17 208 and anti-TfR IgG3-C_{H}3-Av, indicates that there also will not be any significant toxicity associated with in vivo use of anti-TfR IgG3-C_{H}3-Av. Previous clinical studies using potent toxins chemically conjugated to Tf have shown that the cytotoxicity was mainly directed to the tumor cells and that side effects of the treatment were minor or absent, suggesting that anti-TfR IgG3-C_{H}3-Av also will not have unwanted side effects.

### EXAMPLE 2

### Anti-Rat TfR IgG3-C_{H}3-Av Induces Apoptosis in Rat Myeloma Cell Line Y3-Ag1.2.3

Methods

An anti-rat TfR IgG3-C_{H}3-Av fusion protein in accordance with the present invention was constructed in the same manner as in Example 1. Rat myeloma cell line Y3-Ag1.2.3 (5×10⁴ cells/well in DMEM 5% CS) were incubated with 9 nM of the anti-rat TfR IgG3-C_{H}3-Av on a 96-well plate (Becton Dickinson Labware, Franklin Lakes, N.J.) for 24 or 48 hours at 37° C. Twenty-four hours after incubation, cells were harvested and stained with Alexa fluor 488 annexin V and propidium iodine following a procedure suggested by the manufacturer using the Vybrant™Apoptosis Assay Kit #2 (Molecular Probes Inc., Eugene, Oreg.). Forty-eight hours after the incubation, cells were labeled using the APO-BrdU™TUNEL Assay Kit (Molecular Probes Inc.). The cells were fixed with 1% paraformaldehyde and 70% ethanol, followed by DNA labeling with terminal deoxynucleotidyl transferase (TdT) and 5-Bromo-2′-deoxyuridine 5′-triphosphate (BrdUTP). The cells were then treated with Alexa Fluor 488 dye-labeled anti-BrdU monoclonal antibody and analyzed by flow cytometry.

Results

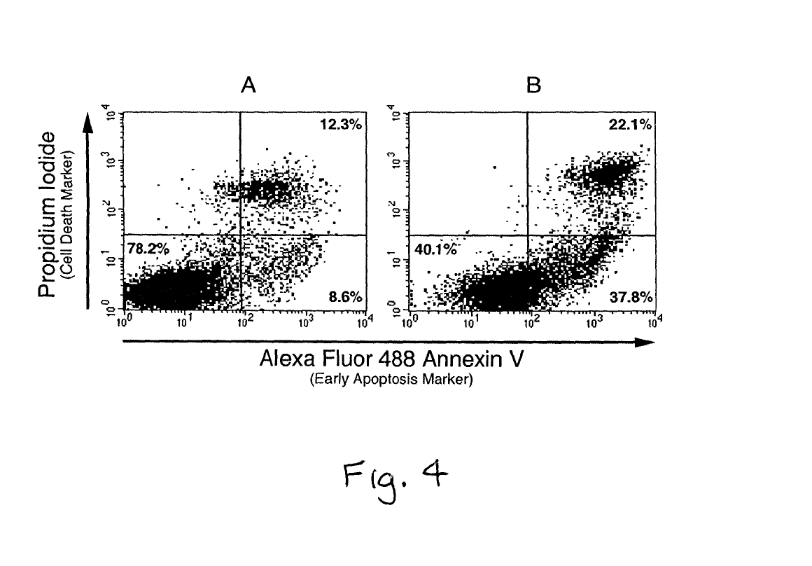

When a cell is undergoing apoptosis, one of the earliest events is the translocation of phosphatidylserine (PS) from the inner to the outer leaflet of the plasma membrane, thus exposing PS to the external cellular environment and to the high affinity binding by annexin V (5). Propidium iodide (PI) is impermeant to live cells and apoptotic cells, but stains dead cells with red fluorescence. Therefore, when a population of cells is incubated with both Alexa Fluor 488 annexin V and PI, annexin V positive, PI negative population represents cells that are alive and undergoing apoptosis; PI single positive and annexin V, PI double positive populations represent dead cells; double negative population represents the healthy cells. The data obtained from this example indicated that there are more apoptotic cells and dead cells in anti-rat TfR IgG3-C_{H}3-Av treated group (FIG. 4B) than in the control group (FIG. 4A). This demonstrates that the antibody fusion protein has a cytotoxic effect on Y3-Ag1.2.3 cells by inducing apoptosis.

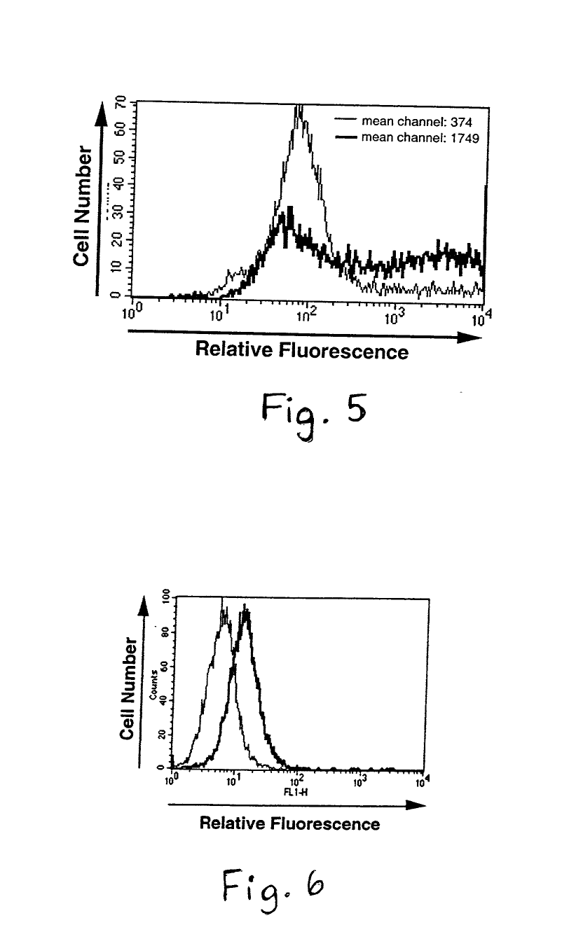

In addition, anti-rat TfR IgG3-C_{H}3-Av induced apoptosis can be demonstrated by another assay. A landmark of apoptosis is the activation of nucleases that degrade the nuclear DNA into small fragments (6). The DNA breaks expose a large number of 3′-hydroxyl ends that can serve as starting points for TdT to add BrdUTP at the 3′ end of the DNA fragment. Fluorochrome labeled anti-BrdUTP antibody can then be added to identify cells with DNA fragmentation. As shown in FIG. 5, Y3-Ag1.2.3 treated with anti-rat TfR IgG3-C_{H}3-Av has significant levels of DNA fragmentation when compared with the control cells. This confirms that the antibody fusion protein in accordance with the present invention has the ability to induce apoptosis in the cell line.

### EXAMPLE 3

### Anti-Human TfR IgG3-C_{H}3-Av Binds Specifically to Transferrin Receptor (TfR) Expressed on the Human Erythroleukemia Cells K562

Experimental Methods

The anti-human TfR IgG3-C_{H}3-Av fusion protein was constructed by the substitution of the variable region of anti-dansyl (5-dimethylamino naphthalene 1-sulfonyl chloride) IgG3-C_{H}3-Av fusion heavy chain (1) with the variable region of the heavy chain of anti-human TfR IgG1 monoclonal antibody 128.1 (7). It was expressed with the mouse/human k light chain gene with the variable region of 128.1 in the murine myeloma P3X63Ag8.653 (8).

Anti-dansyl IgG3-C_{H}3-Av or anti-TfR IgG3-C_{H}3-Av were allowed to complex with biotinylated FITC (b-FITC) in 50 mM Tris base, 150 mM NaCl, pH 7.8 for 3 hours at room temperature. Then, b-FITC alone, or the two antibody fusion proteins complexed with b-FITC were incubated with 10⁶ human erythroleukemia cells K562 (9) for 3 hours on ice. The cells were then washed and incubated for an additional 1 hour on ice, resuspended in 2% paraformaldehyde in PBS, pH 7.4 and analyzed by flow cytometry using a FACScan (Becton-Dickinson, Mountain View, Calif.) equipped with a blue laser excitation of 15 mW at 488 nm.

Results

The anti-human TfR IgG3-C_{H}3-Av specifically binds to the TfR expressed on the human erythroleukemia cells K562 cells (FIG. 6). The isotype-matched specificity control, recombinant anti-dansyl IgG3-C_{H}3-Av, did not bind (FIG. 6) and showed similar fluorescence intensity as cells treated with buffer (PBS) alone (data not shown). Thus, TfR IgG3-C_{H}3-Av is able to simultaneously bind TfR (TfR of K562 cells) and biotynylated compounds (b-FITC).

### EXAMPLE 4

### Direct Antiproliferative Effect of Anti-Human TfR-avidin Fusion Protein on Human Erythroleukemia Cell Line

Experimental Methods

Human erythroleukemia cell line, K562 (5000 cells/well in DMEM 5% CS) were treated with buffer (50 mM Tris base, 150 mM NaCl, pH 7.8) alone or the concentration of antibody fusion protein described in the figure on a 96-well plate (Becton Dickinson Labware, Franklin Lakes, N.J.) for 72 hours at 37° C. The cells were then cultured in 4 mCi/mL of [methyl-³H]-thymidine (ICN Biomedicals, Inc., Irvine, Calif.) for another 24 hours before being harvested onto glass fiber filters using 11050 Micro Cell Harvester, (Skratron, Norway) and counted in a 1205 Betaplate® Liquid Scintillation Counter (WALLAC Inc., Gaithersburg, Md.).

Results

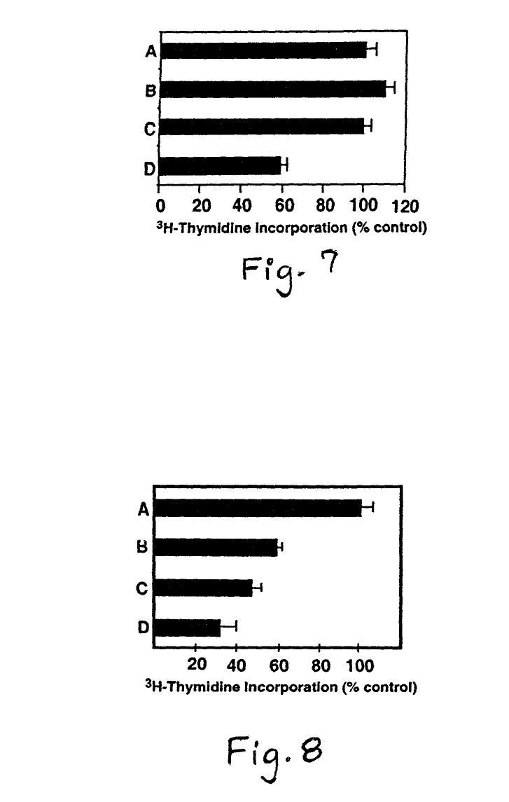

FIG. 7 shows that the anti-human TfR IgG3-C_{H}3-Av inhibits the growth of human erythroleukemia cell line K562 (p<0.001 Student's t-test) as compared with the buffer control. In contrast, mouse anti-human TfR IgG1, which shares the same variable region as anti-human TfR IgG3-C_{H}3-Av does not inhibit. Anti-dansyl IgG3-C_{H}3-Av also does not show an inhibitory activity. Therefore, this result demonstrates that the antiproliferative effect of anti-human TfR IgG3-C_{H}3-Av requires both the variable region and the avidin moiety.

### EXAMPLE 5

### Dose-Dependent Antiproliferative Effect of Anti-Human TfR-avidin Fusion Protein on Human Erythroleukemia Cell Line

Experimental Methods

Human erythroleukemia cell line K562 (5000 cells/well in DMEM 5% CS) were treated with buffer (50 mM Tris base, 150 mM NaCl, pH 7.8) alone or the concentration of antibody fusion protein shown in FIG. 8 where A=buffer; B=25.9 nM; C=51.9 nM; and D=104 nM. The cells were treated on a 96-well plate (Becton Dickinson Labware, Franklin Lakes, N.J.) for 72 hours at 37° C. The cells were then cultured in 4 μCi/mL of [methyl-³H]-thymidine (ICN Biomedicals, Inc., Irvine, Calif.) for another 24 hours before being harvested onto glass fiber filters using 11050 Micro Cell Harvester (Skatron, Norway) and counted in a 1205 Betaplate® Liquid Scintillation Counter (WALLAC Inc., Gaithersburg, Md.).

Results

Anti-human TfR IgG3-C_{H}3-Av significantly inhibits the growth of human erythroleukemia cell line K562 (p<0.001 Student's t-test as compared with the buffer control) in a dose-dependent manner (FIG. 8).

As can be seen from the above examples, the fusion proteins in accordance with the present invention are useful cytotoxic agents which are capable of inducing apoptosis and/or inhibiting cell proliferation. It should be noted although the preceding examples are limited to fusion proteins based on anti-transferrin receptor antibodies, a wide variety of other targeting moieties are possible.

Having thus described exemplary embodiments of the present invention, it should be noted by those skilled in the art that the within disclosures are exemplary only and that various other alternatives, adaptations and modifications may be made within the scope of the present invention. Accordingly, the present invention is not limited to the above preferred embodiments and examples, but is only limited by the following claims.

### BIBLIOGRAPHY

1. Shin S U, Wu D, Ramanathan R, Pardridge W M, and Morrison S L. Functional and pharmacokinetic properties of antibody-avidin fusion properties. J Immunol. 1997, 158 (10):4797-804.

2. Jeffried W A, Brandon M R, Williams A F, Hunt S V. Analysis of lymphopoietic stem cells with a monoclonal antibody to the rat transferrin receptor. Immunology. 1985, 54:331-341.

3. Penichet M L, Kang Y S, Pardridge W M, Morrison S L, Shin S U. An antibody-avidin fusion protein specific for the transferrin receptor serves as a delivery vehicle for effective brain targeting: initial applications in anti-HIV antisense drug delivery to the brain. J. Immunol. 1999, 163:4421-4426.

4. Galfr G, Milstein C, Wright B. Rat x rat hybrid myelomas and a monoclonal anti-Fd portion of mouse IgG. Nature 1979, 277:131-133.

5. Van Engeland M, Nieland L J, Ramaekers F C, Schutte B, Reutelingsperger, C P. Annexin V-affinity assay: a review on an apoptosis detection system based on phosphatidyllserine exposure. Cytometry 1998, 31:1-9.

6. Arends M J, Morris R G, Wyllie A H. Apoptosis. The role of the endonuclease. Am. J. Pathol. 1990, 136:593-608.

7. Friden P M, Olson T S, Obar R, Walus L R, and Putney S D. Characterization, receptor mapping and blood-brain barrier transcytosis of antibodies to the human transferrin receptor. J. Pharmacol. Exp. Ther. 1996, 278:1491-8.

8. Kearney J F, Radbruch A, Liesegang B, and Rajewsky K. A new mouse myeloma cell line that has lost immunoglobulin expression but permits the construction of antibody-secreting hybrid cell lines. J. Immunol. 1979,123:1548-50.

9. Andersson L C, Nilsson K, and Gahmberg C G. K562—a human erythroleukemic cell line. Int. J. Cancer. 1979, 23:143-7.

10. Green N M. Advances in Protein Chemistry, Vol. 29, pp. 85-133, 1975.

11. Kaplan, I I. American Journal of Medical Science, Vol. 207, pp. 733-743, 1944.

12. Gope M L et al. Nucleic Acids Research, Vol. 15, pp.3595-3606, 1987.

13. U.S. Pat. No. 6,287,792 B1.

14. PCT Publication WO 01/07084 A1

15. U.S. Pat. No. 5,672,683

16. U.S. Pat. No. 5,807,715

17. Kang Y S and Pardridge W M. Use of Neutral Avidin Improves Pharmokinetics and Brain Delivery of Biotin Bound to an Avidin-Monoclonal Antibody Conjugate. J. Pharmacology and Experimental Therapeutics, Vol. 29, pp. 344-350,1994.

18. Adamson P J, Zola H, Nicholson I C, Pilkington G, Hohmann A. Antibody against CD20 in patients with B cell malignancy. Leuk Res. 2001, 25: 1047-50.

19. Barth S. hIL-13-PE38QQR. NeoPharm. Curr Opin Investig Drugs. 2001, 2:1309-13.

20. Brooks D, Taylor C, Dos Santos B, Linden H, Houghton A, Hecht T T, Kornfeld S, Taetle R. Phase la trial of murine immunoglobulin A antitransferrin receptor antibody 42/6. Clin Cancer Res. 1995, 1: 1259-65.

21. Habelshaw, H. A., Lister, T. A., and Stansfeld, A. G. s. Correlation of transferrin receptor expression with histological class and outcome in non-Hodgkin lymphoma. Lancet. 1983, 1: 498-500.

22. Hall W A. Targeted toxin therapy for malignant astrocytoma. Neurosurgery. 2000, 46: 544-51.

23. Laske D W, Youle R J, Oldfield E H. Tumor regression with regional distribution of the targeted toxin TF-CRM107 in patients with malignant brain tumors. Nat Med. 1997, 3:1362-8.

24. Leng J, Lang J, Shen K, Guo L. Overexpression of p53, EGFR, c-erbB2 and c-erbB3 in endometrioid carcinoma of the ovary. Chin Med Sci J. 1997, 12: 67-70.

25. Li J Y, Sugimura K, Boado R J, Lee H J, Zhang C, Duebel S, Pardridge W M. Genetically engineered brain drug delivery vectors: cloning, expression and in vivo application of an anti-transferrin receptor single chain antibody-streptavidin fusion gene and protein. Protein Eng 1999,12:787-96.

26. Livingston R B, Esteva F J. Chemotherapy and herceptin for HER2 (+) metastatic breast cancer: the best drug? Oncologist. 2001, 6: 315-6.

27. Manzke O, Russello O, Leenen C, Diehl V, Bohlen H, Berthold F. Immunotherapeutic strategies in neuroblastoma: antitumoral activity of deglycosylated Ricin A conjugated anti-GD2 antibodies and anti-CD3xanti-GD2 bispecific antibodies. Med Pediatr Oncol. 2001, 36:185-9.

28. Marttila A T, Laitinen O H, Airenne K J, Kulik T, Bayer E A, Wilchek M, Kulomaa MS.Recombinant NeutraLite avidin: a non-glycosylated, acidic mutant of chicken avidin that exhibits high affinity for biotin and low non-specific binding properties. FEBS Lett. 2000, 467: 31-6.

29. Mayers, G L, Razeq, J, and Abu-Hadid, M M. Cytotoxic drug conjugates for treatment of neoplastic disease. U.S. Pat. No. 5,393,737. 1995.

30. Mayers, G L, Raghavan, D, Hitt, S, and Glaves, D. Transferrin-Gemcitabine conjugates: application to chemotherapy. In Proceedings of the 89th Annual Meeting of the American Association for Cancer Research, New Orleans, La., USA, Mar. 28-Apr. 1, 1998. p 63. 1998.

31. Meyer D L, Schultz J, Lin Y, Henry A, Sanderson J, Jackson J M, Goshorn S, Rees A R, Graves S S. Reduced antibody response to streptavidin through site-directed mutagenesis. Protein Sci. 2001, 10: 491-503.

32. Nichols J, Foss F, Kuzel T M, LeMaistre C F, Platanias L, Ratain M J, Rook A, Saleh M, and Schwartz G. Interleukin-2 fusion protein: an investigational therapy for interleukin-2 receptor expressing malignancies. Eur. J. Cancer. 1997, 33 Suppl 1: S34-36.

33. Penichet M L, Kang Y S, Pardridge W M, Morrison S L, Shin S U. An antibody-avidin fusion protein specific for the transferrin receptor serves as a delivery vehicle for effective brain targeting: initial applications in anti-HIV antisense drug delivery to the brain. J Immunol. 1999a, 163: 4421-6.

34. Penichet M L, Shin S U, and Morrison S L. Fab fusion proteins: Immunoligands. In Antibody Fusion Proteins. Chamow S. M. and A. Ashkenazi, eds. John Wiley & Son, Inc., New York. 1999b. pp.15-52.

35. Penichet M L and Morrison S L. Antibody Engineering. In Encyclopedia of Molecular Medicine (EMM). Thomas E. Creighton, ed. John Wiley & Son, Inc., New York, 2001. Volume 1, pp. 214 to 216.

36. Prost A C, Menegaux F, Langlois P, Vidal J M, Koulibaly M, Jost J L, Duron J J, Chigot J P, Vayre P, Aurengo A, Legrand J C, Rosselin G, Gespach C. Differential transferrin receptor density in human colorectal cancer: A potential probe for diagnosis and therapy. Int J Oncol. 1998, 13: 871-5.

37. Psarras, K., Ueda, M., Yamamura, T., Ozawa, S., Kitajima, M., Aiso, S., Komatsu, S., and Seno, M. s. Human pancreatic RNase1-human epidermal growth factor fusion: an entirely human ‘immunotoxin analog’ with cytotoxic properties against squamous cellcarcinomas. Protein Eng. 1998, 11: 1285-92.

38. Rybak, S. M., Saxena, S. K., Ackerman, E. J., and Youle, R. J. s. Cytotoxic potential of ribonuclease and ribonuclease hybrid proteins. J. Biol. Chem. 1991, 266: 21202-7.

39. Shinohara H, Fan D, Ozawa S, Yano S, Van Arsdell M, Viner J L, Beers R, Pastan I, Fidler I J. Site-specific expression of transferrin receptor by human colon cancer cells directly correlates with eradication by antitransferrin recombinant immunotoxin. Int J Oncol. 2000, 17: 643-51.

40. Strauchen, J. A. and Breakstone, B. A.s. IL-2 receptor expression in human lymphoid lesions. Immunohistochemical study of 166 cases. Am. J. Pathol. 1987, 126: 506-512,

41. Sweeney E B and Murphy J. R. Diphtheria toxin-based receptor-specific chimaeric toxins as targeted therapies. Essays Biochem. 1995, 30: 119-31.

42. Singh M, Atwal H, Micetich R. Transferrin directed delivery of adriamycin to human cells. Anticancer Res 1998,18: 1423-1427.

What is claimed is:

1. A method for inducing apoptosis in cells, said method comprising the step of exposing one or more cells to a cytotoxic agent for a sufficient time and at a sufficient temperature to induce apoptosis of said one or more cells, said cytotoxic agent consisting of a targeting moiety and an avidin moiety wherein said targeting moiety is capable of binding to one or more of said cells.

2. A method for inducing apoptosis in cells according to claim 1 wherein said cells are liquid or solid tumor cells

3. A method for inducing apoptosis in cells according to claim 2 wherein said liquid or solid tumor cells are cancerous.

4. A method for inducing apoptosis according to claim 1 wherein said targeting moiety binds to a cell surface protein or carbohydrate.

5. A method for inducing apoptosis in cells according to claim 1 wherein said targeting moiety is capable of binding to one or more growth factor receptors located on said cells.

6. A method for inducing apoptosis in cells according to claim 1 wherein said cells are in vivo.

7. A method for inducing apoptosis in cell according to claim 1 wherein said cells are in vitro.

8. A method for inducing apoptosis in cells according to claim 1 wherein said targeting moiety comprises an antibody, antibody fragment, scFv or a ligand.

9. A method for inducing apoptosis in cells according to claim 1 wherein said avidin moiety comprises molecules selected from the group consisting of avidin and avidin analogues.

10. A method for inducing apoptosis in cells according to claim 8 wherein said avidin moiety comprises two molecules selected from the group consisting of avidin and avidin analogues.

11. A method for inducing apoptosis according to claim 1 wherein said cytotoxic agent is a fusion protein.

12. A method for inhibiting the proliferation of a proliferating cell population, said method comprising the step of exposing said cell population to a cytotoxic agent for a sufficient time and at a sufficient temperature to inhibit proliferation of said proliferating cell population, said cytotoxic agent consisting of a targeting moiety and an avidin moiety wherein said targeting moiety is capable of binding to one or more of said cells.

13. A method for inhibiting the proliferation of a cell population according to claim 12 wherein said cell population comprises liquid or solid tumor cells.

14. A method for inhibiting the proliferation of a cell population according to claim 13 wherein said liquid or solid tumor cells are cancerous.

15. A method for inhibiting proliferation of a cell population according to claim 12 wherein said targeting moiety binds to a cell surface protein or carbohydrate.

16. A method for inhibiting the proliferation of a cell population according to claim 12 wherein said targeting moiety is capable of binding to one or more growth factor receptors located on said cells.

17. A method for inhibiting the proliferation of a cell population according to claim 12 wherein said cell population is in vivo.

18. A method for inhibiting the proliferation of a cell population according to claim 12 wherein said cell population is in vitro.

19. A method for inhibiting the proliferation of a cell population according to claim 12 wherein said targeting moiety comprises an antibody, antibody fragment, scFv or a ligand.

20. A method for inhibiting the proliferation of a cell population according to claim 12 wherein said avidin moiety comprises molecules selected from the group consisting of avidin and avidin analogues.

21. A method for inhibiting the proliferation of a cell population according to claim 12 wherein said avidin moiety comprises two molecules selected from the group consisting of avidin and avidin analogues.

22. A method for inhibiting the proliferation of a cell population according to claim 12 wherein said cytotoxic agent is a fusion protein.

23. A composition for use in treating cells to induce apoptosis and/or inhibit cell proliferation wherein said cells include cell surface proteins or carbohydrates, said composition comprising:
a cytotoxic agent consisting of a targeting moiety and an avidin moiety wherein said targeting moiety is capable of binding to one or more of said cell surface proteins or carbohydrates; and
a pharmaceutically acceptable carrier.

24. A composition for use in treating cells to induce apoptosis and/or inhibit cell proliferation according to claim 23 wherein said targeting moiety comprises an antibody, antibody fragment, scFv or ligand.

25. A composition for use in treating cells to induce apoptosis and/or inhibit cell proliferation according to claim 23 wherein said avidin moiety comprises molecules selected from the group consisting of avidin and avidin analogues.

26. A composition for use in treating cells to induce apoptosis and/or inhibit cell proliferation according to claim 23 wherein said cell surface protein or carbohydrate is a growth factor receptor.

27. A composition for use in treating cells to induce apoptosis and/or inhibit cell proliferation according to claim 24 wherein said antibody is an anti-tranferrin receptor antibody.

28. A composition for use in treating cells to induce apoptosis and/or inhibits cell proliferation according to claim 23 wherein said targeting moiety is a fusion protein.
